(19) United States
(12) Patent Application Publication
(10) Pub. No.: US 2023/0194549 A1
(43) Pub. Date: Jun. 22, 2023

(54) METHOD FOR DETERMINING THE RISK OF INCIDENCE OF A CARE-ASSOCIATED INFECTION IN A PATIENT

(71) Applicants: BIOMÉRIEUX, Marcy-l'Etoile (FR); BIOASTER, Lyon (FR); HOSPICES CIVILS DE LYON, Lyon (FR)

(72) Inventors: François MALLET, Villeurbanne (FR); Guillaume MONNERET, Lyon (FR); Virginie MOUCADEL, Sassenage (FR); Alexandre PACHOT, Sulignat (FR); Estelle PERONNET, Lyon (FR); Julien TEXTORIS, Villeurbanne (FR); Fabienne VENET, Lyon (FR); Thomas RIMMELÉ, Lyon (FR)

(73) Assignees: BIOMÉRIEUX, Marcy-l'Etoile (FR); BIOASTER, Lyon (FR); HOSPICES CIVILS DE LYON, Lyon (FR)

(21) Appl. No.: 17/918,184

(22) PCT Filed: Apr. 8, 2021
(86) PCT No.: PCT/FR2021/050615

§ 371 Date: Mar. 2, 2023

(30) Foreign Application Priority Data
Apr. 9, 2020 (FR) FR2003591

(51) Int. Cl. G01N 33/68; G01N 33/569

(52) U.S. Cl. CPC G01N 33/6866; G01N 33/56938; G01N 2333/31; G01N 2333/52; G01N 2800/26; G01N 2800/50

(57) ABSTRACT

An in vitro or ex vivo method, based on the measurement of the expression of cytokine(s), from a patient's blood sample, incubated with a stimulus, for determining the risk of occurrence of a healthcare-associated infection in the patient, within seven days following the day on which the collection of the biological sample has been performed from the patient.

The present invention concerns an in vitro or ex vivo method, based on the measurement of the expression of cytokine(s), from a patient's blood sample, incubated with a stimulus, for determining the risk of occurrence of a healthcare-associated infection in said patient, within seven days following the day on which the collection of the blood sample has been performed from said patient.

The development of healthcare-associated infections is a major complication related to medical care, particularly in medical care structures such as hospitals (where we will more specifically talk about nosocomial infections). It has been demonstrated that nosocomial infections in intensive care units, which occur in 20 to 40% of patients, have been associated with increased morbidity and mortality, a longer duration of need for organ failure supportive care, longer hospital stays, higher healthcare costs, and a considerable use of antibiotics, contributing to antimicrobial resistance. The apparition of healthcare-associated infections has been particularly exacerbated in recent years, due to the increase in multi-resistant pathogens. The World Health Organization (WHO) estimates the number of nosocomial infections in hospitals in Europe at about 5 million, leading to about 50,000 deaths and an additional annual cost of 13 to 24 billion euro. Many factors influence the occurrence and development of healthcare-associated infections, such as the patient's general state of health, but also factors related to patient management (e.g. the administration of antibiotics and/or the use of invasive medical devices), factors related to the hospital environment (e.g. the ratio of the number of nurses to the number of patients), and the variable use of aseptic techniques by the hospital staff. Recommendations have been published, and the establishment of infection control programs has been encouraged, in particular by the US Department of Health and Human Services, the European Center for Disease Prevention and Control, the WHO and the national agencies, for which the prevention and reduction of healthcare-associated infections have become a major priority. It has been demonstrated that healthcare-associated infection control programs turn out to be particularly effective in reducing severe infections. However, it has been estimated that a maximum of 65 to 70% of cases of blood and urinary tract infections, related to the placement of catheters, and of 55% of cases of pneumonia associated with mechanical ventilation and infections at the surgical site, could be avoided. Moreover, the observance and application of procedures according to the recommendations might be complicated in some hospitals, especially in low- and middle-income countries. The early identification of patients at risk of developing a healthcare-associated infection would be a key step in the prevention of these infections and the management of these patients. According to some models, an assay that would reduce the time to identify patients at high risk of contracting healthcare-associated infections would reduce mortality in these patients, with a good cost/effectiveness ratio. However, there is currently no clinical in vitro diagnostic assay for identifying patients at high risk of contracting a healthcare-associated infection.

Functional assays, or Immune Functional Assays (IFA), are assays that measure directly, ex vivo, the ability of one or more cell population(s) to respond to a stimulus with which the cells are brought into contact. These functional assays have for example been used in research to study the anergy of monocytes, most often by measuring TNFα at the protein level after ex vivo stimulation with lipopolysaccharide (LPS), as well as in the clinic, in the case tuberculosis, by measuring interferon γ (IFNγ) at the protein level after stimulation with an antigen from Mycobacteria tuberculosis. Functional assays were also used as part of a study aimed at defining the limits of a normal immune response (i.e. in a “healthy” context) in response to different infectious challenges (Urrutia et al (2016), Cell Reports 16: 2777-2791).

In addition, it has previously been shown, in patients with sepsis after major visceral surgery, that the secretion of IL2, TNFα and partly IFNγ by T lymphocytes, stimulated with anti-CD3/CD28, was significantly reduced in non-surviving patients, compared to surviving and control patients; however, no link with the risk of contracting a healthcare-associated infection was mentioned (Heidecke et al (2000), Chirurg. 71(2): 159-65).

On the other hand, some studies have shown that the level of cytokines secreted into blood samples from pediatric patients (in septic shock, with severe injuries or having received cardiac surgery) and previously incubated with a stimulus, showed some differences between patients who developed a nosocomial infection and those who did not (Muszynski et al (2014), Crit Care 18: R145; Muszynski et al (2014), Shock 42(4): 313-321; Greathouse et al (2018), Circulation 138: A16525). The methods used made it possible to assess either innate immune function (using LPS as a stimulus) or adaptive immune function (using phytohaemagglutinin (PHA) as a stimulus). LPS binds to the TLR4 receptor, more particularly at the level of innate immunity cells, while PHA is a lectin that binds to T lymphocytes. However, these studies generally refer to the occurrence of a healthcare-associated infection within fourteen to thirty days following the day of the aggression (i.e. onset of septic shock, injury or surgery), whereas an earlier prediction would be preferable, because the decision in terms of therapeutic intervention must be taken quickly, preferably within a week. There is therefore a need to develop assays making it possible to determine the risk of occurrence of a healthcare-associated infection in a patient, in the week following the day on which the sample was collected from this patient, i.e. within seven days following the day on which this collection has been performed.

Yet, it has been discovered that, quite surprisingly, functional assays based on the measurement of the expression of cytokine(s), from a blood sample of a patient, incubated with some types of stimulus (such as superantigens which simultaneously bind cells of innate immunity and cells of adaptive immunity), allow determining the risk of occurrence of a healthcare-associated infection in this patient, within seven days following the day on which the collection of the blood sample has been performed from this patient These patients at risk of developing a healthcare-associated infection could advantageously benefit from an individualized management or an immunostimulatory treatment.

Thus, an object of the present invention is an in vitro or ex vivo method for determining the risk of occurrence of a healthcare-associated infection, preferably a nosocomial infection, in a patient, preferably a patient in a healthcare facility, within seven days following the day on which the collection of the blood sample has been performed from said patient, comprising:

a) A step of incubating a blood sample of the patient with a stimulus, said stimulus comprising a molecule selected from the group consisting of:
a molecule capable of binding at least one type of antigen-presenting cell (APC), preferably a type of cell of the innate immunity, and at least one type of cell of the adaptive immunity (or acquired immunity), preferably a T lymphocyte,
one or several molecule(s) allowing direct activation of T lymphocytes, said one or several molecule(s) being selected from antibodies and antibody analogs, and
a molecule of the imidazoquinoline type;

b) A step of measuring the expression, from the stimulated blood sample resulting from step a), of at least one cytokine, said cytokine being produced by innate immunity cells and/or by adaptive immunity cells.

Of course, the method comprises a final step of determining the risk of occurrence of said infection.

In the context of the present invention:
The term «patient» refers to an individual (human being) who has come into contact with a healthcare professional, such as a doctor (for example, a general practitioner) or a medical structure or a health facility (for example, a hospital, and more particularly the emergency unit, the resuscitation unit, an intensive care unit or an on-going care unit, or a medical structure for the elderly, of the nursing home type). The patient may be, for example, an elderly person, as part of a vaccination protocol (in particular in a nursing home or even with a general practitioner);
An infection is called «healthcare-associated», if it occurs during or after a (diagnostic, therapeutic, palliative, preventive, educational or surgical) management of a patient by a healthcare professional, and if it has been neither present nor incubating at the start of treatment. Healthcare-associated infections (HAIs) comprise infections developed within a healthcare facility (known as nosocomial infections) but also during healthcare delivered outside this setting. When the infectious state at the start of treatment is not specifically known, a delay of at least 48 hours or a delay greater than the incubation period is commonly accepted to define a HAI. For surgical site infections, infections occurring within thirteen days of the surgery or, if an implant, prosthesis or prosthetic material are placed in the year following the surgery, are usually considered to be healthcare-associated.
A «blood sample» refers to a sample of whole blood or a cell sample derived from blood (i.e. a sample obtained from blood and containing at least one type of cell, such as a sample of peripheral blood mononuclear cells or PBMC);
By «antigen-presenting cell» (APC), it should be understood a cell of the immune system which presents portions of intruders to T lymphocytes. It may be an innate immune cell (e.g. monocyte, macrophage, dendritic cell) or a B lymphocyte;
By «antibody analog», it should be understood a molecule mimicking paratope/epitope recognition behavior, such as with antibodies. These antibody analogs are well known to those skilled in the art. Examples include Fab, Fab′, F(ab′)₂, scFv, nanobodies, adnectins/monobodies, diabodies, affibodies, aptamers, anticalines, DARPins, avimers, affilins, fynomers, nanofitins/affitins, knottins, pronectins, Kunitz domains.

A healthcare-associated infection occurring within seven days following the day on which the collection of the blood sample has been performed from the patient, may occur during the 1^{st}, 2^{nd}, 3^{rd}, 4^{th}, 5^{th}, 6^{th}, or 7^{th} day following the day on which the collection of the blood sample has been performed from the patient (regardless of the day on which this sample collection has been performed).

Preferably, in the method as previously described:
the patient is a patient in a healthcare facility, preferably in a hospital, more preferably a patient in the emergency unit, a resuscitation unit, the intensive care unit or on-going care unit; more preferably, a patient suffering from a severe inflammatory attack; more preferably, a patient in a septic state (and more particularly, a patient in a septic shock and in particular a patient in need of vasopressors and/or whose lactate exceeds 2 mmol/L), a patient suffering from burns (and more particularly, severe burns), a patient suffering from trauma (and more particularly, severe trauma), or a patient undergoing surgery (and more particularly, major surgery).

In this case, the method according to the invention allows determining the risk of occurrence of a nosocomial infection in patient. By a septic patient (or patient with sepsis), it should be understood a patient with at least one life-threatening organ failure caused by an inappropriate host response to an infection. By septic shock, it should be understood a sepsis subtype, in which hypotension persists, despite adequate vascular filling. In the case of a patient in a septic state (already suffering from a first infection), the method according to the invention makes it possible to determine the risk of occurrence of a secondary infection.

Patients may be pediatric patients (18 years old or younger) or adult patients (over 18 years old); preferably, they are adult patients.

Preferably, the method as previously described, in all its embodiments, is applied to a blood sample containing leukocytes, in particular containing at least T lymphocytes. The blood sample can for example be a sample of purified T lymphocytes. It can also be a sample of peripheral blood mononuclear cells (or PBMC), which is made up of lymphocytes (B, T and NK cells), dendritic cells and monocytes, and which is generally obtained by the Ficoll method, well known to those skilled in the art. However, it will be preferable to directly use a sample of whole blood (that is to say containing all of the leukocytes, erythrocytes, platelets and plasma), as collected by the venous route (for example by using tubes containing an anticoagulant), in order to minimize manipulations of the sample and to preserve the physiological cellular interactions between the different cell populations involved in the immune response, and to better reflect the complexity of the innate and adaptive immune responses in the patient. In particular, while PBMCs only contain mononuclear cells, whole blood also contains granulocytes (or polymorphonuclear cells).

The collection of the blood sample may have been performed, if necessary, on admission to a healthcare facility or after the patient's evolution, in particular during the first week (i.e. on D1, D2, D3, D4, D5, D6 or D7) after the inflammatory attack or after admission.

The method according to the invention is based on a functional assay, in which a patient's blood sample is incubated with a stimulus. In particular, this stimulus binds specifically to an adaptive immunity cell (more particularly, a T lymphocyte, in particular at the level of the alpha and beta variable chains of the receptors of T cells or TCR) and possibly also to an antigen-presenting cell (in particular at the level of the major histocompatibility complex of class II or MHC II).

Thus, in the method as previously described, in all its embodiments, the stimulus may for example comprise one (several) molecule(s) capable of binding:
at least one type of antigen-presenting cell (APC), said APC possibly being in particular a type of cell of innate immunity (e.g. a monocyte, a macrophage, or a dendritic cell) or a type of cell of the adaptive immunity (e.g. a B lymphocyte), on the one hand, and
at least one type of adaptive immunity cell (such as a T lymphocyte), on the other hand.

Preferably, said molecule is capable of binding at least one type of antigen-presenting cell (APC) and at least one type of the adaptive immunity cell, simultaneously, so as to form a «bypass» between these cells.

Preferably, it is a stimulus comprising a molecule of the superantigen type or a molecule analogous to superantigen. Superantigens are toxins of a protein nature, capable of stimulating a large number of T lymphocytes, through their simultaneous binding to the β chain of the variable domain (V_{β}) of a T cell receptor via the hypervariable region CDR4, and to a molecule of MHC II (class II major histocompatibility complex), present on the surface of an antigen-presenting cell (APC). The forced interaction that is established between the antigen-presenting cell carrying the MHC and the T lymphocytes whose T cell receptor carries the V_{β} segment, causes a polyclonal activation of these T lymphocytes, independently of their specificity for the presented peptide antigen. When a stimulus comprising a molecule of the superantigen type is used, the blood sample used in the method according to the invention preferably contains T lymphocytes and antigen-presenting cells. Among the superantigens of more particular interest, mention may in particular be made of the superantigens produced by staphylococcal species and the superantigens produced by streptococcal species. Staphylococcal and streptococcal superantigens are known. Examples of staphylococcal superantigens include SEA, SEB, SEC (including in particular the SEC1, SEC2, SEC3 and SEC4 variants), SED, SEE, SEF, SEG, SEH, SEI, SEJ, SEK, SEL, SEM, SEN, SEO, SEP, SEQ, SER, SES, SET, SEU, SEV (Staphylococcal Enterotoxin A to V) and TSST-1, and examples of streptococcal superantigens include SPE A, SPE B and SPE C (Streptococcal Pyogenic Exotoxin A to C). Preferably, the stimulus comprises at least one molecule selected from SEA (Staphylococcal Enterotoxin A), SEB (Staphylococcal Enterotoxin B) and SEC (Staphylococcal Enterotoxin C). Among the molecules analogous to a superantigen, mention may be made, for example, of bispecific antibodies, capable of binding on the one hand to a T lymphocyte, and on the other hand to an antigen-presenting cell (such as, for example, antibodies capable of binding on the one hand to V_{β} on T lymphocytes, and on the other hand to a molecule of MHCII or to a TLR-type receptor, on antigen-presenting cells).

In the method according to the invention, it is also possible to use a stimulus comprising one or several antibodies (or antibody analog(s)) allowing direct activation of the T lymphocytes, in particular via the recognition and activation of a receptor on the surface of the T lymphocyte. These may be antibodies that are physically and/or chemically associated with each other, more preferably still by coupling on polymers, by coupling on beads, by coupling on BSA (Bovine Serum Albumin) or by coupling between them. Preferably, they may be anti-CD3 antibodies (such as Muromonab-CD3, marketed under the name Orthoclone OKT3), which bind to T lymphocytes, more preferably said anti-CD3 antibodies being associated physically and/or chemically with one or several antibodies, which can preferably be selected from the list consisting of: anti-HLA-DR (Human Leukocyte Antigen-DR) antibodies, anti-CD28 antibodies, anti-CD2 antibodies and/or anti-CD137/TNFRSF9 antibodies, which bind to antigen-presenting cells, as previously indicated.

In the method according to the invention, it is also possible to use a stimulus comprising a molecule of the imidazoquinoline type (structural analogues of a nucleoside, including a ring in their structure, of low molecular weight), preferably a TLR receptor agonist, more preferably a TLR7 and/or TLR8 receptor agonist. This type of stimulus produces in vivo antiviral and antitumor effects. An example of an imidazoquinoline-type stimulus, Resiquimod (R848), which binds to human TLR7 and TLR8 on dendritic cells, or more generally on antigen-presenting cells (NF-KB-dependent response) may be mentioned. Direct effects on T lymphocytes have also been described (Smits et al (2008), Oncologist 13(8): 859-875).

It is particularly advantageous to use, as a part of the method according to the invention, systems allowing a standardization of the procedures; in particular, it is possible to use semi-closed culture systems (e.g. tubes) pre-filled with the culture medium and the stimulus of interest, which are standardized, e.g. which contain a well-defined stimulus (i.e. without inter-batches at the level of the production of the stimulus, as to its nature/composition) and/or loaded in «batch», so as to control the quantity of stimulus in the tube and to have tube-to-tube reproducibility. Preferably, these tubes can also allow the collection of the blood sample (which allows the cells to be stimulated at the time of collection), and more preferably, they allow the collection of a precise volume of blood. An example of standardized systems are TruCulture® tubes.

In the method as described above, in all its embodiments, the step of incubating the patient's blood sample with the stimulus can be carried out at different temperatures (preferably at 37° C.) and at different incubation times (preferably between 1 hour and 48 hours of incubation; for example with an incubation of 1 hour or less, 2 hours or less, 4 hours or less, 12 hours or less, 24 hours or less, or 48 hours or less). Short incubation times are particularly advantageous for the implementation of the assay in clinical practice.

The incubation of the patient's blood sample with a stimulus, as previously described, induces a cellular response resulting in the production of cytokine(s), which allows determining the risk of occurrence of a healthcare-associated infection in a patient, within seven days following the day on which the collection of the blood sample has been performed from said patient. Thus, in the method as previously described, in all its embodiments, the expression of at least one cytokine is measured, said cytokine being produced by cells of innate immunity and/or by cells of the adaptive immunity. A low level of expression of cytokine(s) is associated with a higher risk of contracting a healthcare-associated infection within seven days following the day on which a collection of the blood sample has been performed. This may include measuring the expression of at least one, at least two, at least three, at least four, at least five, at least six, at least seven, at least eight, at least nine, at least ten cytokine(s) selected from the group consisting of GM-CSF, IFNγ, IL2, IL3, IL4, IL5, IL6, IL10, IL17 and TNFα (“adaptive immunity” list), preferably selected from the group consisting of GM-CSF, IFNγ, IL2, IL3, IL4, IL5, IL6, IL10 and IL17, more preferably selected from IFNγ and IL2. These cytokines correspond to cytokines produced by adaptive immunity cells (in particular, T lymphocytes). It may also include measuring the expression of at least one, at least two, at least three, at least four, at least five, at least six, at least seven, at least eight, at least nine, at least ten, at least eleven, at least twelve, at least thirteen, at least fourteen cytokine(s) selected from the group consisting of CCL2 (MCP1), CCL3 (MIP1 alpha), CCL4 (MIP1 beta), CXCL8 (IL8), CXCL10 (IP10), IFNγ, IL1α, IL1β, IL1RA, IL3, IL6, IL10, IL18 and TNFα (“innate immunity” list), more preferably said at least one cytokine being IFNγ. These cytokines correspond to cytokines produced by innate immunity cells (in particular, monocytes and/or macrophages).

It may also include (in particular when the stimulus comprises a molecule capable of binding at least one type of antigen-presenting cell and at least one type of adaptive immunity cell) measuring the expression of at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen different cytokines, respectively selected from the “innate immunity” list and from the “adaptive immunity” list, preferably at least one cytokine among the at least two different cytokines being selected from IFNγ and IL2, more preferably said two different cytokines being IFNγ and IL2.

The measurement of the expression of cytokine(s) can in particular be carried out at the protein level. The techniques allowing such measurement of protein expression are well known to those skilled in the art. By way of examples, mention may be made of assays by immunoassays, such as ELISA (Enzyme Linked ImmunoSorbent Assay), ELFA (Enzyme Linked Fluorescent Assay) and RIA (radio immunoassays), by ECL (Electrochimiluminescence) and assays by mass spectrometry.

Preferably, the method as previously described, in all its embodiments, can also comprise a step of measuring the expression, from a control blood sample without stimulation (that is to say the sample blood being incubated under the same conditions as the stimulated blood sample, but in the absence of stimulus) of the same cytokine(s) as that/those measured from the stimulated blood sample. More preferably, the method can also comprise a step of calculating the ratios of the expression of each cytokine in the stimulated blood sample, compared to the expression of the same cytokine in the control blood sample (non-stimulated), or a step of subtracting the expression of each cytokine in the control (unstimulated) blood sample from the expression of the same cytokine in the stimulated blood sample.

Preferably, in the method as previously described, in all its embodiments, the expression of the cytokine(s) in the stimulated blood sample of the patient is compared with a reference value or with the expression of the same cytokine(s) in a reference blood sample. The reference blood sample can be, for example, a blood sample from a volunteer (healthy individual), a mixture of samples from several volunteers (healthy individuals), a sample from a patient or a mixture of samples from several patients, the patient(s) possibly in particular having suffered from a severe inflammatory attack, as described above; these reference blood samples preferably being stimulated by the same stimulus as the stimulated blood sample of the patient to be tested.

Preferably, in the method as previously described, in all its embodiments, the ratio of the expression of each cytokine in the stimulated blood sample, relative to the expression of the same cytokine in the control blood sample (unstimulated), is compared with a reference value corresponding to the ratio of the expression of the same cytokine in a stimulated sample from the reference blood sample, relative to the expression of the same cytokine in an unstimulated sample from the reference blood sample.

Preferably, in the method as previously described, in all of its embodiments, the value resulting from the subtraction of the expression of each cytokine in the control (unstimulated) blood sample from the expression of the same cytokine in the stimulated blood sample is compared with a reference value resulting from the subtraction of the expression of the same cytokine in an unstimulated sample from the reference blood sample with the expression of the same cytokine in a stimulated sample from the reference blood sample.

The functional assay according to the invention can be used alone, or else combined with some immune parameters, so as to improve the prediction of the occurrence of a healthcare-associated infection within seven days following the day on which the collection of the blood sample has been performed. Thus, the method as previously described, in all its embodiments, may also comprise a step of measuring, in a patient's blood sample that has not been incubated with a stimulus, the concentration of IL10, the concentration of IL6, the number of molecules HLA-DR per monocyte, the percentage of CD10^{low}/CD16^{low} neutrophils and/or the percentage of CD10^{high}/CD16^{high} neutrophils.

The invention also relates to the use:
of means for detecting the expression of at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen cytokine(s) (in particular selected from those described above), said detection means being preferably antibodies, or
of a kit comprising such detection means, said kit preferably further comprising a stimulus as defined above,
   to determine the risk of occurrence of a healthcare-associated infection, preferably a nosocomial infection, in a patient, preferably a patient in a healthcare facility, within seven days following the day on which the collection of the blood sample, from which the expression of cytokine(s) is measured, has been performed from said patient.

### FIGURES

FIG. 1: Effect of different stimulus (SEB, SEC and a bifunctional conjugate ‘aHLADR/aCD3’ including anti-HLA-DR and anti-CD3 monoclonal antibodies grafted onto BSA) on the secretion of cytokines IL6, IL10 and CXCL10. The incubation of 100 μL (panel 1) or 300 μL (panel 2) of whole blood with the stimulus was carried out at 37° C. for 24 h (panel 1) or 16 h (panel 2), before measuring the quantity of secreted cytokines. “NS”: statistically non-significant.

The present invention is illustrated without limitation by the following examples.

### EXAMPLE 1

### Materials and Methods

A prospective, longitudinal and monocentric observational clinical study has been carried out at the Edouard Herriot Hospital (Lyon, France). The design of this clinical study has been published in Rol et al. (2017), BMJ Open 7(6): e015734. The clinical study was approved by the National Agency for the Safety of Medicines and Health Products (ANSM) in November 2015 and the South-East II Personal Protection Committee in December 2015. Amendments to the protocol were made in July 2016, then in January 2017. In brief, a total of 377 patients, in a septic state (n=35) or in septic shock (n=72), suffering from severe burns (n=24), severe trauma (n=137) or hospitalized in a resuscitation unit or intensive care unit after major surgery (n=109), and 175 healthy volunteers have been included between December 2015 and March 2018.
Patients in septic state/in septic shock: according to the first clinical protocol, only patients in septic shock have been included, on the basis of a suspicion of an infectious focus, a start of treatment with catecholamines within 48 hours following admission to the resuscitation unit and of treatment with catecholamines (noradrenaline)>0.25 μg/kg/min for at least 2 hours. Then, the eligibility criteria were modified in August 2016, following the publication of a new definition of septic shock, Sepsis 3 (Singer et al. (2016), JAMA 810-801: (8)315). The patients in septic shock have therefore been included on the basis of a suspicion of an infectious focus, a start of treatment with catecholamines within 48 hours following admission to resuscitation unit and of vasopressor therapy necessary to maintain blood pressure 65 mm Hg and lactate concentration>2 mmol/L (18 mg/dL), despite the correction of hypovolaemia. In 2017, the possibility was added to include patients in a sepsis state (according to the Sepsis 3 definition), namely the suspicion of an infectious focus and the increase in the SOFA score 2 points compared to the basic SOFA within 48 hours following admission to the resuscitation unit. For this population, day 1 corresponds to the day of diagnosis of sepsis or septic shock;
Severe trauma: in the first protocol, only patients with severe trauma have been included (Injury Severity Score (ISS)≥25). In August 2016, the possibility was added to also include less severe injuries (16<ISS<24). For this population, day 1 corresponds to the day of admission to the resuscitation unit or intensive care unit (˜trauma day);
Major surgery: in the first protocol, only esogastrectomy, Bricker-type bladder resection, cephalic duodenopancreatectomy and surgery of the abdominal aorta by laparotomy have been considered. Other types of surgery with a high risk of complication were added in January 2017: (total or caudal) pancreatectomy, neuroendocrine tumors, hepatectomy (on the right side), extended colectomy (laparotomy), abdoperineal resection, nephrectomy (laparotomy, PKD), ilio-femoral bypass (Scarpa). For this population, day 1 corresponds to the day of surgery;
Severe burns: the patients have been selected on the basis of a total surface area of burns greater than 30%. For this population, day 1 corresponds to the day of admission to the resuscitation unit or intensive care unit (˜day of the burn).

The exclusion criteria have mainly related to factors that could have impacted the immune status and biased the results (for example: severe neutropenia, corticosteroid treatments, onco-haematological pathology, etc.). Each event leading to a suspected healthcare-associated infection occurring in the hospital before day 30 has been independently reviewed by three physicians not involved in the recruitment of the patients. Twenty-six percent of the patients have developed at least one healthcare-associated infection before day 30, or before leaving the hospital.

Heparinized whole blood samples were collected several times for the patients, i.e. 3-4 times in the first week (on days 1 or 2: D1/2, on days 3 or 4: D3/4 and on days 5, 6 or 7: D5/7), then 3 times at later times (around D14, D28 and D60). These samples were dispensed into preheated TruCulture tubes (Myriad Rbm, Austin, Tex., USA), containing either medium alone (“control sample”) or medium with SEB (100 ng/mL) (“stimulated sample”). These tubes were then inserted into a dry block incubator and maintained at 37° C. for 24 hours. After incubation, the concentrations of IFNγ or IL2 were measured by an ELISA assay (References: SPCKB-CS-000292 and SPCKB-CS-000955, Biotechne, respectively), using the nanofluidic platform ELLA (ProteinSimple, San José, Calif., USA), as recommended by the supplier.

Regarding the data analysis, the association between cytokine secretion and the risk of occurrence of a healthcare-associated infection was assessed for different time intervals of infection occurrence (i.e. time between sample collection and the first occurrence of an infection). The different time periods considered were: healthcare-associated infection within 4 days and within 7 days after sample collection, regardless of when the sample was collected. For each patient who has developed a healthcare-associated infection (i.e. “case patients”), the sample considered corresponds to the closest sample collection (with a minimum delay of 24 hours) before the occurrence of the first episode of healthcare-associated infection. For the patients who did not develop a healthcare-associated infection (i.e. “control patients”), a matching method was used to select, for each case patient, a control patient with the same sample collection day, and close SOFA and Charlson scores. Finally, a single control was selected for each unique case. Univariate logistic regressions were implemented. The analysis was made for all types of patients combined. The association between cytokine secretion and the occurrence of healthcare-associated infection was estimated in the form of Odds Ratios expressed as inter-quartile distance (OR IQR) with the 95% confidence interval associated therewith.

### Results

It has been observed that a lower concentration of IL2 or IFNγ after stimulation with SEB was associated with a higher risk of occurrence of a healthcare-associated infection within 4 or 7 days following the day of sample collection. (Table 1).

TABLE 1
Association between the measurement of IFNγ or IL2 after
stimulation by SEB and the risk of occurrence of a healthcare-
associated infection within 4 or 7 days following the day of sample
collection. The Odd Ratios (OR IQR) are expressed as inter-quartile
distance with the associated 95% confidence interval (CI).

| | Number of days after sample | | |
|---|---|---|---|
| | collection until first | | |
| Measured | occurrence of healthcare- | | |
| Cytokine | associated infection | OR IQR (IC) | P |
| IFNγ | 4 days | 0.65 (0.44-0.88) | 0.014 |
| | 7 days | 0.63 (0.43-0.86) | 0.009 |
| IL2 | 4 days | 0.51 (0.30-0.82) | 0.007 |
| | 7 days | 0.65 (0.42-0.96) | 0.038 |

### EXAMPLE 2

In this example, the inventors compared the effect of three stimulus (SEB, SEC and a bifunctional conjugate ‘aHLADR/aCD3’ including anti-HLA-DR and anti-CD3 monoclonal antibodies grafted onto BSA) on the secretion of different cytokines (IL6, IL10 and CXCL10).

### Materials and Methods

Whole blood samples (100 μL or 300 μL) from healthy volunteers (n=5 for each condition) were incubated at 37° C. for 16 h or 24 h in the presence of the stimulus at a concentration of approximately 1.4 10⁻⁸ M (i.e. 0.0004 g/L of SEB (TruCulture®, Myriad, ref 782-001124), 0.0004 g/L of SEC (Toxin Technology, Inc., ref CT111-SEC1) or 0.0094 g/L of aHLADR/aCD3 conjugate (Ultra-LEAF Purified anti-human HLA-DR-NA.41 (Ozyme), ref BLE307666 and Ultra-LEAF Purified anti-human CD3-NA.41 (Ozyme), ref BLE317347)) in RPMI. After incubation, the concentrations (in pg/mL) of the cytokines (IL6, IL10, CXCL10—ProteinSimple) were measured using the ELLA nanofluidic platform (ProteinSimple). Comparisons between different stimulus were made using a non-parametric test t (Wilcoxon signed rank test on paired samples). A value of p≤0.05 was considered statistically significant (two-sided test).

### Results

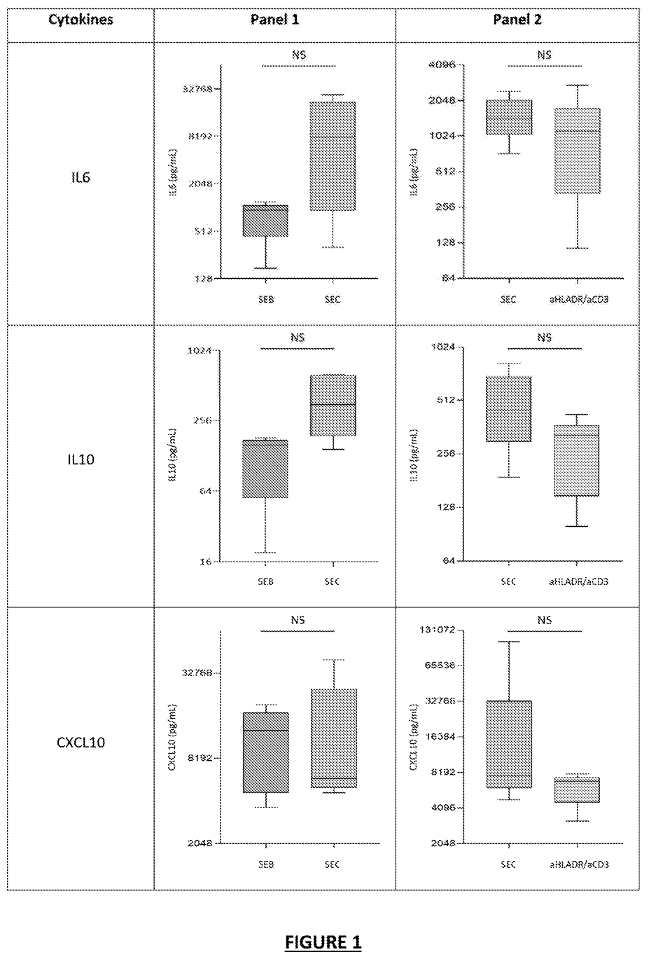

FIG. 1 shows the results obtained with the different stimulus. Panel 1 illustrates that there was no statistically significant difference in the level of IL6, IL10 and CXCL10 secretion between a stimulation by SEB and a stimulation by SEC. Similarly, panel 2 shows that there is no statistically significant difference in the level of secretion of these cytokines between a stimulation by SEC and a stimulation by the aHLADR/aCD3 conjugate. These three stimulus therefore induce the secretion of similar amounts of cytokines in blood samples.

1. An in vitro or ex vivo method for determining the risk of occurrence of a healthcare-associated infection in a patient within seven days following the day on which the collection of the blood sample has been performed from the patient, comprising:
a) a step of incubating a blood sample of the patient with a stimulus, the stimulus comprising a molecule selected from the group consisting of:
a molecule capable of binding at least one type of antigen-presenting cell (APC), and at least one type of cell of the adaptive immunity,
one or several molecule(s) allowing direct activation of T lymphocytes, the one or several molecule(s) being selected from antibodies and antibody analogs, and
a molecule of the imidazoquinoline type;
b) a step of measuring the expression, from the stimulated blood sample resulting from step a), of at least one cytokine, the cytokine being produced by innate immunity cells and/or by adaptive immunity cells.

2. The method according to claim 1, wherein the patient is a patient within a healthcare facility.

3. The method according to claim 1, wherein the patient is an adult patient, over the age of 18 years.

4. The method according to claim 1, wherein the blood sample is a whole blood sample.

5. The method according to claim 1, wherein the stimulus comprises a molecule capable of binding at least one type of antigen-presenting cell (APC), and at least one type of adaptive immunity cell, the molecule being a molecule of superantigen type.

6. The method according to claim 1, wherein the stimulus comprises a molecule selected from the superantigens produced by staphylococcal species and the superantigens produced by streptococcal species.

7. The method according to claim 1, wherein the stimulus comprises SEA (Staphylococcal Enterotoxin A), SEB (Staphylococcal Enterotoxin B) or SEC (Staphylococcal Enterotoxin C).

8. The method according to claim 1, wherein the stimulus comprises a molecule capable of binding at least one type of antigen-presenting cell (APC) and at least one type of adaptive immunity cell, the molecule being a molecule analogous to a superantigen.

9. The method according to claim 1, wherein the stimulus comprises one or several antibodies allowing a direct activation of the T lymphocytes, the one or several antibodies being selected from antibodies recognizing and activating a receptor on the surface of the T lymphocyte.

10. The method according to claim 1, wherein the stimulus comprises a molecule of the imidazoquinoline type.

11. The method according to claim 1, wherein in step b), the expression of at least one cytokine selected from the group consisting of GM-CSF, IFNγ, IL2, IL3, IL4, IL5, IL6, IL10, IL17 and TNFα (“adaptive immunity” list) is measured.

12. The method according to claim 1, wherein in step b), the expression of at least one cytokine selected from IFNγ and IL2 is measured.

13. The method according to claim 1, wherein in step b), the expression of at least one cytokine selected from the group consisting of CCL2 (MCP1), CCL3 (MIP1 alpha), CCL4 (MIP1 beta), CXCL8 (IL8), CXCL10 (IP10), IFNγ, IL1α, IL1β, IL1RA, IL3, IL6, IL10, IL18 and TNFα (“innate immunity” list).

14. The method according to claim 1, wherein in step b), the expression of at least two different cytokines, respectively selected from the “innate immunity” list and from the list “adaptive immunity”, is measured.

15. The method according to claim 14, wherein one of the at least two different cytokines is selected from IFNγ and IL2.

16. The method according to claim 1, wherein the expression of cytokine(s) is measured at the protein level.

17. The method according to claim 1, wherein the expression of cytokine(s) is measured by ELISA (Enzyme Linked ImmunoSorbent Assay), ELFA (Enzyme Linked Fluorescent Assay), RIA (radio immunoassays), ECL (Electrochemiluminescence) or mass spectrometry.

18. The method according to claim 1, wherein it comprises a step of measuring the expression, from a control blood sample without stimulation, of the same cytokine(s) than that/those measured from the stimulated blood sample.

19. The method according to claim 18, wherein it comprises a step of calculating the ratios of the expression of each cytokine in the stimulated blood sample, relative to the expression of the same cytokine in the control blood sample.

20. The method according to claim 1, wherein it further comprises a step of measuring, in a blood sample from the patient which has not been incubated with a stimulus, the concentration of IL10, the concentration of IL6, the number of molecules of HLA-DR per monocyte, the percentage of CD10^{low}/CD16^{low} neutrophils and/or the percentage of CD10^{high}/CD16^{high} neutrophils.

21. A method comprising applying the means for detecting the expression of at least one cytokine as defined in claim 1, the detection means being antibodies, or
of a kit comprising such detection means as defined in claim 1,
to determine the risk of occurrence of a healthcare-associated infection in a patient, within seven days following the day on which the collection of the blood sample, from which the expression of cytokine(s) is measured, has been performed from the patient.
